SEQUENCE LISTING (24 sequences)

SEQ ID NO: 1 - DNA, 1065 bp, Malus domestica
  gene (1)..(1065): Malus domestica mRNA for C-type MADS-box protein (MdMADS14)
acccacttcccacttctgcaattcttccttccggttgccaagtgcaaccccaaaagaaaaactcaaagtcaagaactaacagaaagagccacaattcatctattttgaggggtttttgccatttttcatccttgtaacaatggagttcgcaaatcaagcacctgagagctctacccaaaaaaaattgggaagaggcaaaattgagattaagcggatcgaaaacactaccaatcgacaagtcaccttctgcaaacgccgcaacggattgcttaagaaagcctatgaattgtctgttctttgtgatgctgaagttgctcttatcgtcttctccacccgtggccgcctctatgagtatgctaacaacagcgttagagcaacaatcgacaggtacaaaaaagcatgcgctgattctacggacggtggatctgtatcagaagctaacactcagttttatcagcaggaagcatcaaaactgcgaagacagatccgagaaattcagaattcaaacaggcatatactgggggaatcccttagcaccttgaaagtcaaggaactgaaaaacctagaaggaagattggagaaaggaatcagcagaataagatccaaaaagaatgaaatcctgttttctgaaatcgaattcatgcaaaagagggagactgagctgcaacaccacaacaattttctgagagcaaagatagctgaaagcgagagggaacagcagcagcagcaaacacatatgattccgggaacttcctacgatccgtcgatgccttcgaattcgtatgacaggaacttcttccctgtgatcttggagtccaataataaccattaccctcgccaaggccagacagctctccaacttgtttgaaatgctggactgccgtctgatgttcttctatccatatcctctgatctgtcttcataaatctatgagataattgacgttgtagtttttatgtatatgggagaaccagtttgctcatgttctccataatatatatatgtgtgatgatggaccccaattctgtgataacatatatagtaaattttattttctcaccccga

SEQ ID NO: 2 - DNA, 876 bp, Malus domestica
  gene (1)..(876): Malus x domestica AGAMOUS-like protein mRNA, complete cds (MdMADS16)
gcaattcttccttcccgttgccaagtgcaaccccaatagaaaaactcaaagtcaagaactagctaacagagaaaaccacaattcatcaatttggaggggtttttgccatttttcatccttgcaacaatggagttcccaaatcaagcacccgagagctcctcccagaaaaaattgggaaggggcaaaattgagattaagcggatcgaaaacactacaaatcgacaagttaccttctgcaaacgccgcaacggattgcttaagaaagcctatgaattgtctgttctttgtgatgctgaagttgctcttatcgtgttctccaaccgtggccgcctctatgagtatgctaacaacagtgttagagcaacaatcgacaggtacaaaaaagcatacgctgatcctacgaacagtggatctgtttcagaagccaacactcagttttatcagcaggaagcatccaaactgcgaagacagatccgagaaattcagaattcaaacaggcatatactgggtgaagctcttagctccttgaacgccaaggaactgaagaacctagaaggaagattggagaaaggaatcagcagaataagatccaaaaagaatgaaatgctgttttctgaaatcgaattcatgcaaaaaagggagaccgagctgcaacaccacaacaattttctgagagcaaagatagctgaaaacgagagggaagagcagcagcatacacacatgatgccgggaacttcctacgatcagtcaatgccttcgcattcttatgacaggaacttcctcccagcggtgatcttggagtccaacaataaccattaccctcaccaagtccagacagctctccaacttgtttgaaatgctggactgccgtctgat

SEQ ID NO: 3 - DNA, 20 bp, Artificial Sequence
  first forward degenerate primer
  misc_feature (1)..(20): 6th, 12th, 15th nucleotide ′n′ represent inosine
aaycgncargtnacnttytg

SEQ ID NO: 4 - DNA, 19 bp, Artificial Sequence
  first reverse degenerate primer
  misc_feature (1)..(19): 3th, 12th, 15th and 18th nucleotide ′n′ represent inosine
tcngcgatyttnshnckna

SEQ ID NO: 5 - DNA, 20 bp, Artificial Sequence
  second forward degenerate primer
  misc_feature (1)..(20): 9th and 18th nucleotide ′n′ represent inosine
aaraargcntaygarytntc

SEQ ID NO: 6 - DNA, 36 bp, Artificial Sequence
  third forward primer
tctagaactagtggatcccccgggctgcaggaattc

SEQ ID NO: 7 - DNA, 27 bp, Artificial Sequence
  third reverse primer
atccactgttcgtaggatcagcgtatg

SEQ ID NO: 8 - DNA, 28 bp, Artificial Sequence
  forth forward primer
ggctgcaggaattcggcactaggcaatt

SEQ ID NO: 9 - DNA, 26 bp, Artificial Sequence
  forth reverse primer
gcaagcttatcagacggcagtccagc

SEQ ID NO: 10 - DNA, 21 bp, Artificial Sequence
  MdMADS14 forward primer
gggaacagcagcagcagcaaa

SEQ ID NO: 11 - DNA, 21 bp, Artificial Sequence
  MdMADS14 reverse primer
ctccaagatcacagggaagaa

SEQ ID NO: 12 - DNA, 21 bp, Artificial Sequence
  MdMADS16 forward primer
tgaaaacgagagggaagagca

SEQ ID NO: 13 - DNA, 21 bp, Artificial Sequence
  MdMADS16 reverse primer
caagatcaccgctgggaggaa

SEQ ID NO: 14 - DNA, 21 bp, Artificial Sequence
  ACTIN forward primer
cgatggccaagtcatcacaat

SEQ ID NO: 15 - DNA, 21 bp, Artificial Sequence
  ACTIN reverse primer
tctcatgaatgccagcagctt

SEQ ID NO: 16 - DNA, 249 bp, Artificial Sequence
  hybridization probe
atgcaaaaaagggagaccgagctgcaacaccacaacaattttctgagagcaaagatagctgaaaacgagagggaagagcagcagcatacacacatgatgccgggaacttcctacgatcagtcaatgccttcgcattcttatgacaggaacttcctcccagcggtgatcttggagtccaacaataaccattaccctcaccaagtccagacagctctccaacttgtttgaaatgctggactgccgtctgat

SEQ ID NO: 17 - DNA, 21 bp, Artificial Sequence
  npt II forward primer
gaggctattcggctatgactg

SEQ ID NO: 18 - DNA, 21 bp, Artificial Sequence
  npt II reverse primer
atcgggagcggcgataccgta

SEQ ID NO: 19 - DNA, 24 bp, Artificial Sequence
  MdMADS forward primer
gaattcaaacaggcatatactggg

SEQ ID NO: 20 - DNA, 21 bp, Artificial Sequence
  MdMADS reverse primer
gacggatcgtaggaagttccc

SEQ ID NO: 21 - DNA, 21 bp, Artificial Sequence
  RIN forward primer
tggtacacttgaaggaaccca

SEQ ID NO: 22 - DNA, 20 bp, Artificial Sequence
  RIN reverse primer
catgtgttgatggtgctgca

SEQ ID NO: 23 - DNA, 18 bp, Artificial Sequence
  Le20ox-1 forward primer
cccaacaagcatctgagc

SEQ ID NO: 24 - DNA, 18 bp, Artificial Sequence
  Le20ox-1 reverse primer
ttcctaaggcgagctccg

(19) United States
(12) Patent Application Publication
(10) Pub. No.: US 2007/0250945 A1
(43) Pub. Date: Oct. 25, 2007

(54) Usage of Mad-Box Genes in Fruit & Seed Development by Regulating Active Gibberellin Synthesis

(76) Inventors: Soon-Kee Sung, Kyonggi-do (KR); Young-Pyo Lee, Taejon-si (KR); Gyun-Hee Yu, Kyonggi-do (KR); Yeon-Ok Choi, Taejon-si (KR)

Correspondence Address: LUCAS & MERCANTI, LLP, 475 PARK AVENUE SOUTH, 15TH FLOOR, NEW YORK, NY, 10016, US

(21) Appl. No.: 10/588,095

(22) PCT Filed: Jan. 31, 2005
(86) PCT No.: PCT/KR05/00282

§ 371 Date: Jul. 28, 2006

(30) Foreign Application Priority Data
Feb. 2, 2004 (KR) 10 -2004-0006551
Feb. 17, 2004 (KR) 10-2004-001432

(51) Int. Cl. C12N 15/82; A01H 5/00; A01N 43/00; C12N 5/04; C07H 21/04; C12N 15/00

(52) U.S. Cl. 800/278; 435/320.1; 435/410; 504/209; 536/23.6; 800/298

(57) ABSTRACT

The present invention relates to a novel use of MADS-box genes, particularly, a novel use of MADS-box gene selected from a group consisting of a gene having a nucleotide sequence set forth in SEQ. ID. No 1 containing a nucleotide sequence encoding MADS-domain, a gene having a nucleotide sequence set forth in SEQ. ID. No 2 containing a nucleotide sequence encoding MADS-domain and a gene encoding an amino acid sequence having at least 85% homology within the region other than MADS-domain, for the regulation of fruit and seed development. MADS-box genes of the present invention can be effectively used for the regulation of parthenocarp fruit formation as well as the development of fruit and seed in a plant by controlling active gibberellin synthesis via generating a transgenic plant containing the gene of the present invention.

### TECHNICAL FIELD

The present invention relates to a novel use of a MADS-box gene. In particular, it relates to the use of a MADS-box gene selected from the group consisting of a gene having a nucleotide sequence set forth in SEQ. ID. No 1 containing a nucleotide sequence encoding MADS-domain, a gene having a nucleotide sequence set forth in SEQ. ID. No 2 containing a nucleotide sequence encoding MADS-domain and a gene encoding an amino acid sequence having at least 85% homology within the region other than MADS-domain, for the regulation of fruit and seed development.

### BACKGROUND ART

In the most of angiosperm plants, fruit provides a suitable environment for seed maturation and often a mechanism for the dispersal of mature seeds.

The Fruit development usually starts with development and fertilization of ovary, which occurs by a variety of chemical and physiological changes (Dong Y-H et al., J. Amer. Soc. Hort. Sci., 1997, 122:752-757). After the completion of fertilization, an egg cell develops into an embryo, polar nuclei develop into endosperm and an ovule develops into seed. An ovary develops into fruit by the differentiation and growth of a cell and transformation and accumulation of reserve substances therein. However, there are some cases where not only an ovary but also other parts of a floral organ such as receptacle are involved in the fruit development. Such fruits are called pome fruits and an apple is an example. In general, the development of ovary into fruit is also accompanied by a seed formation, however, in the case of fruits without seed, seed does not form even after the fertilization and only the ovary itself develops. The forming a fruit without seed is called parthenocarpy. Parthenocarpy occurs when ovary develops into a fruit in the absence of pollination (citrus, banana, pineapple, etc) or fertilization of an egg cell after pollination (some orchidaceae), or an embryo is aborted after the fertilization (grapes, peach, cherry etc).

MADS-box genes represents a gene family composing 30 or more amino acid sequences encoding a transcription factor containing a conserved region called MADS-domain. Many of those genes have been shown to be involved in the differentiation of a flower and other various organs in a plant by regulating transcription. Specifically, MADS-box genes are classified into group A, group B and group C, according to their respective functions (ABC model). Genes of group A take responsible for the development of sepal and petal, genes of group B for the development of petal and stamen, and genes of group C for the development of stamen and pistil. Among MADS-box genes derived from Arabidopsis thaliana, APETALA1 (AP1) belongs to group A, APETALA (AP3) and PISTILL (PI) have been confirmed to belong to group B, and AGAMOUS (AG) is classified into group C (Gunter Theissen et al., Plant Molecular Biology, 2000, 42: 115-149). Further, Floral binding protein 7 (FBP7) and floral binding protein 11 (FBP11) which are found to be involved in the development of ovule have been classified into new group D (Gerco C. Angenent et al., Plant Cell, 1995, 7: 1569-1582).

Gibberellin is a class of diterpenoid plant hormone, designated as GA1 to GAn depending on its kind. The basic structure of Gibberellin consists of 4 rings and contains 20 carbon atoms. Gibberellin plays an essential role in the development and growth of a plant, particularly, in the development of seed, stimulation of germination, growth of a stem, generation and growth of a flower, anthocyanin biosynthesis, fruit-setting, etc (Richard Hooley, Plant Mol. Biol., 1994, 26: 1529-1555). The growth promotion by gibberellin is not by stimulating the cell division but by stimulating an elongation and enlargement of a cell. In addition, gibberellin stimulates the formation of flower bud or antithesis. And, it is also used for vernalization and long day treatment (Peter Hedden et al., Annu. Rev. Physiol. Plant Mol. Biol., 1997, 48:431-460).

Such functional activities of gibberellin, for example, in the growth of a plant, germination of a dormant seed, promotion of the growth of a dormant bud and parthenocarp in a plant as mentioned above have been widely utilized in farms to increase productivity by accelerating the growth of an herbaceous plant or to increase the size of a fruit.

During the biosynthesis of Giberellin, monooxygenases synthesize GA₁₂ and GA₅₃ in the early stage of the giberrelin synthesis, which are subsequently converted into C19-GA having physiological activities by GA 20-oxidase and 3β-hydroxylase resulting in the production of biologically active giberreline. Specifically, GA 20-oxidase oxidizes 20^{th} carbons of GA₁₂ and GA₅₃ step by step, providing the backbone structure of C-19 in gibberellin, and 3β-hydroxylase catalyzes 3β-hydroxylation reaction on the C-19 structure of gibberellin thus obtained by GA 20-oxidase, producing biologically active gibberellin (Neil Olszewski et al., Plant Cell, 2002, 14: S61-S80).

The expression of GA 20-oxidase involved in biosynthesis of active gibberellin is negatively feedback regulated by active gibberellin. Precisely, the level of expression of mRNA transcripts for GA 20-oxidase is decreased as the active gibberellin synthesis is increased. On the contrary, the level of expression of mRNA transcripts for GA 20-oxidase is increased as the active gibberellin synthesis is decreased, resulting in the precise regulation of the amount of GA 20-oxidase synthesized (Martin et al., Planta, 1996, 200(2): 159-66; Neil Olszewski et al., Plant Cell, 2002, 14: S61-S80; Victor B. et al., Plant physiol., 2003, 132: 1283-1291). For example, Le20ox-1, a GA 20-oxidase isolated from tomato, converts C-20 gibberellin into C-19 gibberellin that is subsequently activated by 3β-hydroxylase. When the active gibberellin is over-synthesized, the expression of Le20ox-1 mRNA is decreased, by the negative feedback regulation, to reduce the amount of Le20ox-1 enzyme synthesized. Thus, C-19 gibberellin synthesis is reduced with the decrease of the amount of Le20ox-1 enzyme synthesized, resulting in the regulation of the amount of active gibberellin (Mariken Rebers et al., Plant J., 1999, 17(3): 241-250). So, low level expression of Le20os-1 mRNA indicates that active gibberellin is over-synthesized.

It has been reported that RIN gene plays a key role in ethylene synthesis during ripening of tomato fruit. Thus, the decrease of the expression of R#N gene means the decrease of ethylene synthesis during ripening of the fruit. That is, the decrease of ethylene synthesis by the introduced gene results in the delay of ripening (R. C. Herner et al., Plant Physiol., 1973, 52: 38-42; Julia Vrebalov et al., SCIENCE, 2002, 296: 343-346).

At present, the effect of MADS-box genes on the development of fruit and seed has not been fully studied. Although the detailed mechanism of fruit development has yet to be found, it has been shown that several MADS-box genes are expressed in fruits and seeds. It has been recently reported that AGAMOUS-like 15 (AGL15) gene directly controls the expression of gene involved in gibberellin synthesis during seed germination of Arabidopsis thaliana (Huai Wang et al., Plant Cell, 2004, 16: 1206-1219).

Up to now, a number of researchers have developed various transgenic plants by gene manipulation in plants in order to increase the productivity of crops or to develop new cultivars. Further, many transgenic plants that have been proved to be safe have been cultivated and allowed on the market for people to consume. Genetic operation in relation to fruits and seeds has the industrial need, since it can increase the productivity of fruit and seed and be applied to the development of a fruit and a horticultural crop by parthenocarpy.

Thus, the present invention has been completed by isolating novel genes that can regulates the development of fruit and seed by inhibiting or promoting the expression of genes which control the synthesis of active gibberellin, and producing a transgenic plant introduced with genes showing enhanced germination efficiency and parthenocarpic fruit formation.

### DISCLOSURE

### Technical Problem

It is an object of the present invention to provide a novel use of MADS-box gene as a regulator of fruit and seed development.

### TECHNICAL SOLUTION

In order to achieve the above object, the present invention provides a gene that regulates the fruit and seed development by promoting or inhibiting the expression of genes that control the synthesis of active gibberellin. The gene is selected from the group consisting of a gene having a nucleotide sequence set forth in SEQ. ID. No 1 containing a nucleotide sequence encoding MADS-domain, a gene having a nucleotide sequence set forth in SEQ. ID. No 2 containing a nucleotide sequence encoding MADS-domain and a gene encoding an amino acid sequence having at least 85% homology within the region other than MADS-domain.

The present invention also provides an expression vector containing the above gene.

The present invention further provides transgenic plant cells containing the above gene.

The present invention also provides a transgenic plant whose fruit and seed development is regulated, produced by regenerating the above transgenic plant cells by tissue culture technique.

The present invention also provides a method of preparation of the transgenic plant.

The present invention also provides a regulator for fruit and seed development containing the above gene or a vector harboring the above gene as an effective ingredient.

The present invention also provides a regulator for active gibberellin synthesis containing the above gene or a vector harboring the above gene as an effective ingredient.

Hereinafter, the present invention is described in detail.

The present invention provides a gene that can regulates fruit and seed development by promoting or inhibiting the expression of genes that control the synthesis of active gibberellin. The gene is selected from the group consisting of a gene having a nucleotide sequence set forth in SEQ. ID. No 1 containing a nucleotide sequence encoding MADS-domain, a gene having a nucleotide sequence set forth in SEQ. ID. No 2 containing a nucleotide sequence encoding MADS-domain and a gene encoding an amino acid sequence having at least 85% homology within the region other than MADS-domain.

The gene of the present invention that contains a nucleotide sequence set forth in SEQ. ID. No 1 is designated ‘MdMADS14’ gene (FIG. 1), and the gene having a nucleotide sequence set forth in SEQ. ID. No 2 is designated ‘MdMADS16’ gene (FIG. 2). It is preferred that MdMADS16 gene has 90˜99% sequence homology with MdMADS14 gene. Also, MdMADS14 amino acids have at least about 88% sequence homology with MdMADS16 amino acids (FIG. 3).

It has been shown that MdMADS14 gene of the present invention is expressed only in the flowers of an apple tree, but its functions are unknown yet (C. G. van der Linden et al., Journal of Experimental Botany, 2002, 53: 1025-1036). Meanwhile, MdMADS16 gene of the invention is a novel nucleotide sequence having 90˜99% overall homology with MdMADS14 gene (although there is a little difference in homology rate according to a homology comparison program). As shown in FIGS. 1 and 2, both MdMADS16 and 14 genes contain an open reading frame (ORF) coding for 242 amino acids and MADS-domain is located in N-terminal region of each gene. Overall Homology between MdMADS16 and 14 amino acid sequences is 88.4% while the homology within MADS-domain region is 98.3%, and homology within the region other than MADS-domain is at least 85% (FIG. 3).

Although the gene coding an amino acid sequence having at least 85% homology within the region other than MADS-domain of MdMADS14 or MDMADS16 is not represented by a specific sequence list herein, it is easily guessed by a skilled people in this field. MdMADS16 gene was proved to have at least 85% homology within the region other than MADS-domain and have same functions with MdMADS14 gene, indicating that a gene coding an amino acid sequence having over 85% homology in the region other than MADS-domain with the gene of the present invention has equal functions to the gene of the invention.

In the preferred embodiment of the present invention, MdMADS14 or MdMADS16 genes were transferred into a tomato to investigate the function of those genes. From such studies, it has been shown that transformants with over-expressed MdMADS14 gene resulted in two different phenotypes; one was designated MdMADS14 sense 1 and the other MdMADS14 sense 2. In MdMADS14 sense 1, ripening of a fruit was delayed but germination of a seed was promoted. Specifically, as shown in FIG. 8 and FIG. 9, ripening of a fruit in MdMADS14 sense 1 was delayed, compared with that of wild type, while a seed was already germinated in a fruit, suggesting that germination of a seed was much promoted.

Those characteristics were inherited from T1 generation to T2 generation. As shown in FIG. 10, the seeds (T2 generation) of MdMADS14 sense 1 were stored at 4° C. within desiccator for one year, and then germination rate was determined and it was found that germination was promoted in T2 generation of MdMADS14 sense 1, compared with that of wild type. In the case of MdMADS14 sense 2, parthenocarpic fruit was formed and sepal was developed into fruit flesh. Further, anti-sense transformants in which the expression of MdMADS14 gene was suppressed showed the common phenotype of not bearing fruit. The above results indicate that MdMADS14 gene is involved in fruit and seed development.

Transgenic tomato in which MdMADS16 gene was over-expressed resulted in also two different phenotypes corresponding to those shown in transformants over-expressing MdMADS14 gene, each of which was designated MdMADS16 sense 1 and MdMADS16 sense 2. As shown in FIG. 14 ripening of a fruit was delayed in MdMADS16 sense 1, while a seed was germinated inside a fruit, similar to MdMADS14 sense 1 shown above. For MdMADS16 sense 2, as shown in FIG. 15, it showed the same phenotype with MdMADS 14 sense 2 in which sepal was changed into fruit flesh. Further, as shown in FIG. 16, MdMADS16 anti-sense in which the expression of MdMADS16 gene was suppressed did not develop a fruit after pollination, like the case of MdMADS14 anti-sense. The above results indicate that neither MdMADS14 anti-sense nor MdMADS16 anti-sense develop a fruit.

The expressions of RIN gene that is required to promote ethylene synthesis during ripening were investigated in MdMADS14 senses and MdMADS16 senses and were compared with that of wild type. As shown in FIG. 17, the expression level of RIN gene in each of MdMADS14 sense 1 and MdMADS16 sense 1, both having the phenotype of delayed ripening, was decreased.

Moreover, the expression level of a gene coding for LeGA20ox-1 (tomato GA20-oxidase), which can serve as an indicator to indirectly estimate the amount of active giberreline synthesized, in MdMADS14 senses and MdMADS16 senses were compared with that of wild type. As shown in FIG. 18, the expression level of LeGA20ox-1 gene was much decreased in MdMADS14 senses and in MdMADS16 senses than in wild type. Such results indicate that more active gibberellin was synthesized in MdMADS14 senses and in MdMADS16 senses than in wild type. On the contrary, the expression level of LeGA20ox-1 gene was much increased in MdMADS14 anti-sense and in MdMADS16 anti-sense than in wild type, as shown in FIG. 19. Those results indicate that active gibberellin was synthesized much less in MdMADS14 anti-sense and in MdMADS16 anti-sense than in wild type.

From this study, it has been demonstrated that MdMADS14 gene and MdMADS16 gene are highly homologous to each other and are belong to the same gene family. Further, through the analysis of gene functions using the transgenic plants, it has also been demonstrated that those genes were involved in fruit and seed development and have activities of promoting or inhibiting active gibberellin synthesis. In conclusion, MdMADS14 gene and MdMADS16 gene derived from apple in the present invention are novel having the function of regulating fruit and seed development, which are unknown function in MADS-box genes.

The present invention also provides an expression vector containing MdMADS14 gene or MdMADS16 gene.

It is preferred that said gene to be inserted into an expression vector is selected from the group consisting of a gene having a nucleotide sequence set forth in SEQ. ID. No 1 containing a nucleotide sequence encoding MADS-domain, a gene having a nucleotide sequence set forth in SEQ. ID. No 2 containing a nucleotide sequence encoding MADS-domain and a gene encoding an amino acid sequence having at least 85% homology within the region other than MADS-domain.

Any conventional expression vector for plant transformation may be used for the insertion of the gene of the present invention. For the present invention, pGA1530 vector for plant transformation was used for the insertion of MdMADS14 gene (Sung S-K. et al, Plant Physiology, 1999, 120:969-978). The pGA1530 vector contains 35S promoter, T7 terminator, and nptII (neomycin phosphotransfexase), as a marker gene, which provides kanamycin resistance (Stanton B. Gelvin et al, Plant molecular Biology Manual, 1988, A3:1-19). For the insertion of MdMADS16 gene, pCAMBIA 2301 vector for plant transformation was used. The pCAMBIA 2301 vector contains 35S promoter, nos (nopaline synthase) terminator, and nptII (neomycin phosphotransferase), as a marker gene, that provides kanamycin resistance.

In the preferred embodiment of the present invention, expression vectors were constructed by inserting MdMADS14 gene into pGA1530 forwardly or reversely. And the constructed vectors were designated ‘pMdMADS14’ and ‘pMdMADS14-R’. Expression vectors were also constructed by inserting MdMADS16 gene into pCAMBIA 2301 forwardly or reversely, which were designated ‘pMdMADS16’ and ‘pMdMADS16-R’.

The present invention further provides transformed plant cells containing the above gene.

The plant cells that can be used for the introduction of an expression vector of the present invention are not limited to specific forms as long as they can be regenerated into a plant. The cells include, for example, cultured cell buoyant, protoplast, leaf section and callus. An expression vector of the present invention may be introduced into plant cells by one of conventional methods such as polyethylene glycol method, electroporation, Agrobacterium mediating transduction, and particle bombardment, etc. In the preferred embodiment of the present invention, the expression vector was transferred into plant cells by using Agrobacterium containing the gene of the present invention, and in particular, Agrobacterium tumefaciens LBA4404 (A. Hoekema et al., 1983, Nature, 303, 179-181) was used in this invention. Agrobacterium containing the gene of the present invention was prepared by transforming the cell with pMdMADS14 or pMdMADS16, expression vectors of the present invention. Each resulting agrobacterium was designated ‘Agrobacterium tumefaciens LBA4404/pMdMADS14’ and ‘Agrobacterium tumefaciens LBA4404/pMedMADS16’, which were deposited with Korean Collection for Type Cultures (KCTC) of Korea Research Institute of Bioscience and Biotechnology (KRIBB) on Jan. 30, 2004 (Accession Nos: Agrobacterium tumefaciens LBA4404/pMdMADS14; KCTC 10588BP, Agrobacterium tumefaciens LBA4404/pMdMADS16; KCTC 10589BP).

The expression vectors of the present invention can be transferred to any kind of plants that can form a fruit or a seed. Particularly, it is preferred that the plant is selected from a group consisting of food crops such as rice, wheat, barley, corns, soybean, potato, red bean, oat, sorghum; vegetables such as Chinese cabbage, radish, red pepper, strawberry, tomato, watermelon, cucumber, cabbage, melon, pumpkin, spring onion, onion, carrot; industrial crops such as ginseng, Acanthopanax senticosus, tobacco, cotton, sesame, sugar cane, sugar beet, Perilla japonica, peanut, rape; fruits such as apple, pear, orange, jujube, peach, kiwifruit, grapes, tangerine, persimmon, plum, apricot, bananas; floricultural crops such as rose, gladiolus, gerbera, carnation, chrysanthemum, lily, tulip; forage crops such as ryegrass, red clover, orchard grass, alfalfa, tall fescue, perennial ryegrass; fiber crops such as cotton plant; and landscape plants such as flowers and shrubs. It is more preferred that the plant for the introduction of an expression vector of the present invention is tomato plant. In a specific embodiment of the invention, each Agrobacterium transformed with pMdMADS14 or pMdMADS16 was added and incubated with cotyledon sections of minitomato to obtain a transformed tomato cotyledon (cells) containing the expression vector of the present invention.

The present invention also provides a transgenic plant, whose fruit and seed development was controlled, produced by regenerating the above transgenic plant cells by tissue culture technique

Regeneration of the plant cells by tissue culture technique to obtain a transgenic plant of the present invention may be carried out by conventional method of producing transgenic plant. In a specific embodiment of the present invention, tomato cotyledons incubated with agrobacterium having the expression vector containing the gene of the present invention were sub-cultured in a regeneration medium (MS, IAA 1 μM, zeatin 10 μM, sucrose 3%, cefotaxime 350 mg/l, kanamycin 50 mg/l, agar 0.7%). Shoots produced from the cotyledon sections were than collected and it was further incubated in rooting medium (MS ½, IAA 1 μM, sucrose 3%, kanamycin 50 mg/l, agar 0.7%), followed by the selection of a transformant. Untransformed shoots underwent necrosis accompanied by color change while transformed shoots were growing normally with roots in. Shoots with roots were transferred to soil and acclimation. Finally, transgenic tomatoes each with the phenotype characterized by that sepal was changed into fruit flesh and parthenocarpic fruit was formed, with the phenotype showing promoted seed germination and delayed ripening, and with the phenotype characterized by that fruit and seed development was inhibited because ovule was not developed, were prepared.

From the above results, it was confirmed that MADS-box genes of the present invention could be effectively used for the regulation of fruit and seed development and parthenocarpic fruit production in plants by promoting or inhibiting an expression of genes that control the active gibberellin synthesis.

All the plants forming a fruit or a seed can be a target plant of the present invention, which is preferably selected from a group consisting of food crops such as rice, wheat, barley, corns, soybean, potato, red bean, oat, sorghum; vegetables such as Chinese cabbage, radish, red pepper, strawberry, tomato, watermelon, cucumber, cabbage, melon, pumpkin, spring onion, onion, carrot; industrial crops such as ginseng, Acanthopanax senticosus, tobacco, cotton, sesame, sugar cane, sugar beet, Perilla japonica, peanut, rape; fruits such as apple, pear, orange, jujube, peach, kiwifruit, grapes, tangerine, persimmon, plum, apricot, bananas; floricultural crops such as rose, gladiolus, gerbera, carnation, chrysanthemum, lily, tulip; forage crops such as ryegrass, red clover, orchard grass, alfalfa, tall fescue, perennial ryegrass; fiber crops such as cotton plant; and the landscape plants such as flowers and shrubs.

Further, the present invention encompasses the off-springs and clones of the transgenic plants of the present invention as well as fruits, seeds, ears, tubers, tuberous roots, column, callus or protoplasts derived therefrom.

The present invention also provides a preparation method of a plant whose fruit and seed development are regulated, comprising the step of:

A method of preparing a transgenic plant whose fruit and seed development was regulated, comprising the steps of:

1) Constructing an expression vector comprising the gene according to Claim 1;

2) Transferring the vector constructed in Step 1) into Agrobacterium;

3) Co-culturing the transformed Agrobacterium of step 2) with plant tissue; and

4) Regenerating the transformed tissue into a mature transgenic plant.

In step 1) of the preparation method of the present invention, a MADS-box gene that regulates fruit and seed development is preferably selected from the group consisting of a gene having a nucleotide sequence set forth in SEQ. ID. No 1 containing a nucleotide sequence encoding MADS-domain, a gene having a nucleotide sequence set forth in SEQ. ID. No 2 containing a nucleotide sequence encoding MADS-domain and a gene encoding an amino acid sequence having at least 85% homology within the region other than MADS-domain. In step 1), any conventional expression vectors for plant transformation may be used for cloning a MADS-box gene. In a specific embodiment, pGA1530 and pCAMBIA 2301 vectors were preferably used for the insertion of MdMADS14 gene MdMADS16, respectively. In step 2), It is preferred that Agrobacterium which mediates transferring the gene of the present invention into plant cells is Agrobacterium tumefaciens LBA4404 (A. Hoekema et al., 1983, Nature, 303, 179-181). It is preferred that Agrobacterium having an expression vector containing MdMADS14 gene or MdMADS16 gene is ‘Agrobacterium tumefaciens LBA4404/pMdMADS14’ (Accession No: KCTC 10588BP) and ‘Agrobacterium tumefaciens LBA4404/pMdMADS16’ (Accession No: KCTC 10589BP), respectively.

In step 3), all the plants forming a fruit or a seed can be a target plant of the present invention, which is preferably selected from a group consisting of food crops such as rice, wheat, barley, corns, soybean, potato, red bean, oat, sorghum; vegetables such as Chinese cabbage, radish, red pepper, strawberry, tomato, watermelon, cucumber, cabbage, melon, pumpkin, spring onion, onion, carrot; industrial crops such as ginseng, Acanthopanax senticosus, tobacco, cotton, sesame, sugar cane, sugar beet, Perilla japonica, peanut, rape; fruits such as apple, pear, orange, jujube, peach, kiwifruit, grapes, tangerine, persimmon, plum, apricot, bananas; floricultural crops such as rose, gladiolus, gerbera, carnation, chrysanthemum, lily, tulip; forage crops such as ryegrass, red clover, orchard grass, alfalfa, tall fescue, perennial ryegrass; fiber crops such as cotton plant; and landscape plants such as flowers and shrubs.

The present invention also provides a fruit and seed development regulator containing MADS-box gene(s) or an expression vector having the gene(s) as an effective ingredient.

The present inventors analyzed the function of said genes by examining the phenotypes of MdMADS14 senses, MdMADS16 senses and their anti-senses in which the genes were suppressed.

Result from the study showed the following phenotypes: delayed ripening but promoted seed germination (senses 1), change of sepal into fruit flesh and formation of parthenocarpic fruits (senses 2) and inhibited fruit and seed development (anti-senses).

Particularly, for senses 1, germination speed and rate were investigated with seeds (T2 generation) of senses 1. As shown in FIG. 9 and FIG. 10, seed germination was promoted, compared with that of wild type. For senses 2, as shown in FIG. 11, sepal was developed into fruit flesh and parthenocarpic fruit without seed was formed unlike wild type. Plants of anti-senses did not develop fruits, showing that floral organ development in the early stage was not affect (FIGS. 12(a) and (b)) but seed development was inhibited after pollination and so fruits were not formed (FIG. 12(c)).

From the above results, it was confirmed that MdMADS14 gene, MdMADS16 gene and expression vectors containing the genes could be effectively used for the regulation of fruit and seed development in plants

The present invention also provides an active gibberellin synthesis regulator containing MADS-box gene(s) or an expression vector(s) harboring the gene(s) as an effective ingredient.

Le20ox-1 is a GA 20-oxidase isolated from a tomato, whose mRNA synthesis is negatively feedback regulated by active gibberellin. Le20ox-1 is an enzyme that catalyzes the conversion of C-20 gibberellin into C-19 gibberellin, which subsequently activated by 3β-hydroxylase producing active giberellin. When active gibberellin is synthesized in excess, it negatively regulates the mRNA expression of Le20ox-1 by negative feedback, resulting in the decrease in the production of Le20ox-1 enzyme. In turn, C-19 gibberellin synthesis is reduced with the decrease in the mRNA expression of Le20ox-1, resulting in the regulation of active gibberellin synthesis.

The present inventors compared the expressions of Le20ox-1 gene in MdMADS14 senses and MdMADS16 senses in order to investigate the expression levels of the Le20ox-1 gene in transformed tomatoes (FIG. 18 and FIG. 19).

The results show that the expression of Le20ox-1 gene was much decreased in MdMADS14 senses and in MdMADS16 senses than in wild type. On the contrary, the expression of Le20ox-1 gene was increased in MdMADS14 anti-senses and MdMADS16 anti-senses. It clearly indicates that the decrease in the expression of Le20ox-1 gene in MdMADS14 senses and MdMADS16 senses, compared with wild type, is caused by negative feedback by over-expressed active gibberellin, while the increase in the expression of Le20ox-1 gene in MdMADS14 anti-senses and MdMADS16 anti-senses results in the decrease in the active gibberellin synthesis. Therefore, it was confirmed that MdMADS14 and MdMADS16 genes of the present invention and a vector containing those genes could be effectively used for the control of active gibberellin synthesis.

### DESCRIPTION OF DRAWINGS

FIG. 1 is a schematic diagram showing the nucleotide sequence of MdMADS14 gene containing a nucleotide sequence coding for MADS-domain and K-domain as well as the amino acid sequence encoded thereby.

FIG. 2 is a schematic diagram showing the nucleotide sequence of MdMADS16 gene containing a nucleotide sequence coding for MADS-domain and K-domain as well as the amino acid sequence encoded thereby.

FIG. 3 is a schematic diagram showing the homology between amino acid sequences of MdMADS14 and MdMADS16.

FIG. 4 is a phylogenetic tree showing the relation of MdMADS14 and MdMADS16 gene to other MADS-box genes in the same family. AP1: APETALA1 sub-family, SQUA: SQUAMOSA sub-family, AGL2: AGAMOUS-LIKE2 sub-family, AGL6: AGAMOUS-LIKE6 sub-family, AG: AGAMOUS sub-family.

FIG. 5 is a set of graphs showing the results from real-time PCR in which the mRNA level of MdMADS14 and MdMADS16 genes in each developmental stage of leaf, reproductive organ and fruit are quantified and compared.

A: Result of real-time PCR with cDNA from leaves of an apple,

B: Result of real-time PCR with cDNA from flower bud of an immature apple,

C: Result of real-time PCR with cDNA from flower of a mature apple,

D: Result of real-time PCR with cDNA from fruit of an immature apple,

E: Result of real-time PCR with cDNA from fruit of a mature apple,

F: A graph showing the melting temperature of each PCR product, obtained through melting curve analysis during real-time PCR,

G: A graph showing the expression profile of MdMADS14 gene in each organ and in each developmental stage based on the results of graphs A-E,

H: A graph showing the expression profile of MdMADS16 gene in each organ and in developmental stage based on the results of graphs A-E.

a—leaves;

b—flower buds with developing floral organ primordia;

c—flower buds with developing anthers and fused carpels;

d—fruits (7 to 10 days after pollination);

e—ripe fruit.

FIG. 6 is a set of photographs showing the expression profiles of MdMADS16mRNA in floral organ and fruit developmental stages of an apple tree by in situ hybridization.

A: Young flower bud with inflorescence meristem,

B: Young flower bud at the very early stage of differentiation into floral organ having floral primordium,

C: Flower bud in the process of differentiating into floral organ,

D: Immature fruit at 7-10 days after pollination,

c—carpel primordium;

ft—floral tube;

im—inflorescence meristem;

l—leaf appendage;

o—ovule;

st—stamen primordium

FIG. 7 is a photograph showing the result of PCR amplifying nptII gene using genomic DNA of a transformant, in order to confirm the insertion of a recombinant pMdMADS14 plasmid in a transgenic tomatoes.

Lane a: Wild type,

Lane b: MdMADS14 anti-sense,

Lane c: MdMADS14 sense 1,

Lane d: MdMADS14 sense 2

FIG. 8 is a photograph showing the fruit of T1 generation of MdMADS14 sense 1 in which ripening is delayed, compared with wild type.

FIG. 9 is a set of photographs showing the comparison of germination rate between sense 1 seeds (T2 generation), shown in FIG. 8, and wild type seeds, which had been cultured for 5 days under the same conditions.

FIG. 10 is a set of photographs showing the comparison of germination rate between sense 1 seeds (T2 generation), shown in FIG. 8, and wild type seeds. Before the comparison, seeds of both groups were collected and stored in cool dry condition for one year, and then they were cultured for 5 days under the same conditions.

FIG. 11 is a set of photographs showing the result of comparison of fruits between MdMADS14 sense 2 and wild type.

FIG. 12 is a set of photographs showing the results of MdMADS14 anti-sense phenotype analysis. FIG. 12(a) shows the overall appearances of MdMADS14 anti-sense and wild type, FIG. 12(b) is a histological observation of flower and carpel before pollination, and FIG. 12(c) is a histological observation of early fruit and ovule after pollination.

FIG. 13 is a photograph showing the result of PCR amplifying nptII gene using genomic DNA of a transformant, in order to confirm the insertion of a recombinant pMdMADS16 plasmid in a transgenic tomatoes.

Lane a: Wild type,

Lane b: MdMADS16 anti-sense,

Lane c: MdMADS16 sense 1,

Lane d: MdMADS16 sense 2

FIG. 14 is a set of photographs showing that MdMADS14 sense 1 and MdMADS16 sense 1 have the common phenotype of early seed germination inside of a fruit, compared with wild type.

FIG. 15 is a set of photographs showing that MdMADS16 sense 2 and MdMADS14 sense 2 have the common phenotype that sepal is changed into fruit flesh and parthenocarpic fruit is formed as shown in each cross section of fruit.

FIG. 16 is a set of photographs showing overall appearances of MdMADS16 anti-sense and wild type (upper), and a magnified view thereof (lower) indicating no fruit formation.

FIG. 17 is a set of photographs showing the expression levels of RIN gene in the fruits of MdMADS14 senses and MdMADS16 senses, which were quantified by RT-PCR and PCR-Southern blot.

Lane a: Wild type,

Lane b: Fruit of MdMADS14 sense 1,

Lane c: Fruit of MdMADS16 sense 1,

Lane d: Fruit of MdMADS14 sense 2,

Lane e: Fruit of MdMADS16 sense 2

FIG. 18 is a set of photographs showing the expression levels of Le20ox-1 gene in the fruits of MdMADS14 senses and MdMADS16 senses, which were quantified by RT-PCR and PCR-Southern blot.

Lane a: Wild type,

Lane b: Fruit of MdMADS14 sense 1,

Lane c: fruit of MdMADS16 sense 1,

Lane d: Fruit of MdMADS14 sense 2,

Lane e: fruit of MdMADS16 sense 2

FIG. 19 is a set of photographs showing the expression levels of Le20ox-1 gene in leaves of MdMADS14 senses, anti-senses, MdMADS16 senses and anti-senses, which were quantified by RT-PCR and PCR-Southern blot.

Lane a: Wild type,

Lane b: Leaves of MdMADS14 sense 1,

Lane c: Leaves of MdMADS16 sense 1,

Lane d: Leaves of MdMADS14 sense 2,

Lane e: Leaves of MdMADS16 sense 2,

Lane f: Leaves of MdMADS14 anti-sense,

Lane g: Leaves of MdMADS16 anti-sense

### BEST MODE

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Cloning of an MADS-Box Gene from Apple and Nucleotide Sequence Analysis Thereof

cDNA library (SUNG. S. K. et al., Mol. cells, 8: 565-577, 1998) constructed from pistil of apple flower (Malus domestica Borkh cv. Fuji) was used for the preparation of cDNA pool by in vivo excision (Stratagene, USA, Catalog #200450). The nucleotide sequence of MADS-box genes was searched from GenBank (NIH, USA), a database of nucleotide sequences of genes, and then a set of forward and reverse primer was designed from the most conservative region of the nucleotide sequence, which are represented by SEQ. ID. No 3 and SEQ. ID. No 4, respectively. The degenerate PCR was then carried out on pistil cDNA pool, constructed from apple flowers, as a template using the forward and reverse primer set.

The PCR reaction mixture contains 100 ng of cDNA template, 10 μM of each forward and reverse primer, 20 mM of Tris-HCL (pH 8.4), 50 mM of KCl, 1.5 mM of MgCl₂, 0.2 mM of each of dNTP, and 2.5 unit of taq polymerase (Thermus aquaticus DNA polymerase) (TaKaRa, Japan). The PCR reaction was carried out on a thermal cycler (Perkin Elmer 9600, Perkin Elmer) under the following cycling condition: initial denaturation at 94° C. for 3 min; 1 cycle of denaturation, annealing and extension at 94° C./30 seconds, 60° C./1 minute, and 72° C./1 minute, respectively; followed by a touch down PCR where the annealing temperature is decreased by 1° C. every cycle to 50° C. (touchdown annealing temperature) At the last annealing temperature (50° C.), 10 cycles were performed, and final extension at 72° C. for 5 minutes to complete the PCR processes. The resulting PCR product was then diluted 10 fold and used as a template for subsequent nested degenerated PCR using forward primer set forth in SEQ. ID. No 5 and reverse primer set forth in SEQ. ID. No.4 The PCR reaction was carried out under the same condition as above The PCR product was cloned into PGEM-T Easy vector (Promega, USA) and then nucleotide sequence of the product was investigated by using T7 primer and SP6 primer.

The resulting nucleotide sequence data was then analyzed by homology analyzing program (blast, NCBI, USA) and was found to be a partial sequence of the MADS-box gene. Subsequently, a reverse primer set forth in SEQ. ID. No 7 based on the nucleotide sequence information of the above, and a forward primer set forth in SEQ. ID. No 6 based on the nucleotide sequence containing restriction enzyme site of pBluescriptSK(−) vector (Stratagene, USA) were then designed and used for a subsequent PCR reaction. The PCR in the same reaction buffer as above was performed under the following cycling condition; initial denaturation at 94° C. for 3 minutes; 35 cycles of denaturation, annealing and extension at 94° C./30 seconds, 60° C./1 minute, and 72° C./1 minute, respectively; and final extension at 72° C. for 5 minutes to complete the reaction. A 450 bp PCR product thus obtained was then cloned into a pGEM-T Easy vector and the nucleotide sequence of the cloned fragment was investigated by using T7 primer and SP6 primer. The nucleotide sequences data thus obtained were analyzed by homology analyzing program (blast, NCBI, USA) and found to correspond to 5′ end of MADS-box gene having 687 bp. Subsequently, based on the resulting sequence information, a forward primer represented by SEQ. ID. No 8 and reverse primer represented by SEQ. ID. No 9 were designed for a subsequent PCR. The PCR in the same reaction buffer as above was performed under the following cycling condition; initial denaturation at 94° C. for 3 minutes; 35 cycles of denaturation, annealing and extension at 94° C./30 seconds, 60° C./1 minute, and 72° C./1 minute, respectively; and final extension at 72° C. for 5 minutes to complete the reaction. An amplified product about 910 bp in size thus obtained was then cloned into a PGEM-T Easy vector. The resulting plasmid was then used for a transformation of XL1-Blue MRF′, an Escherichia coli strain, and then isolated therefrom.

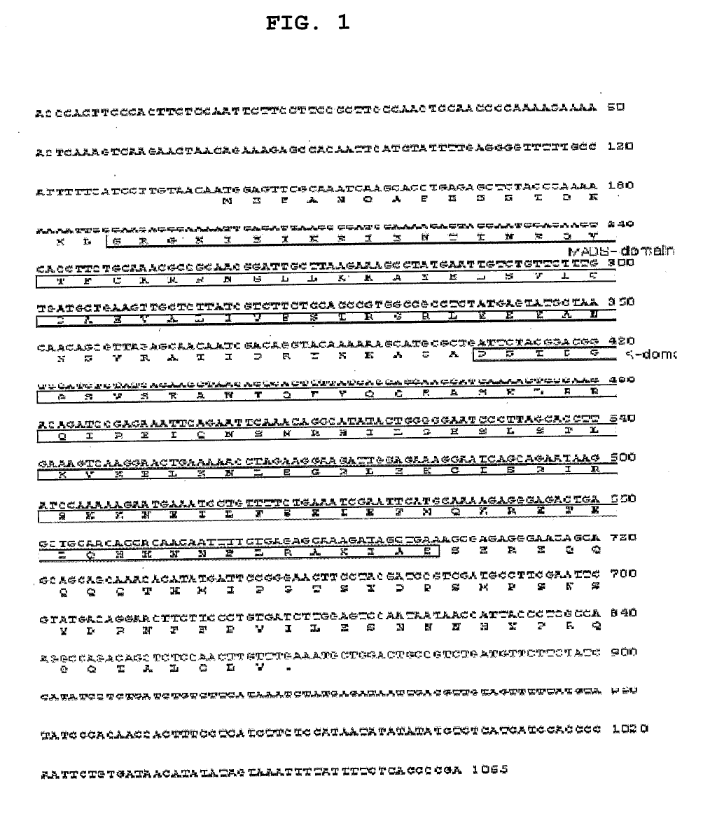

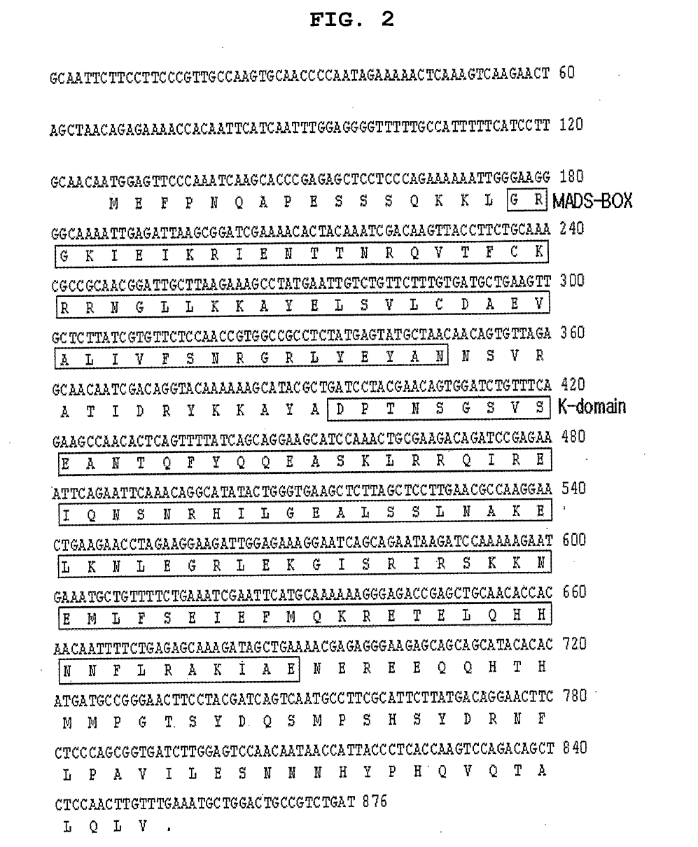

Nucleotide sequence of the isolated plasmid was investigated. As a result, full-length sequences of two different genes, as shown in FIG. 1 and FIG. 2, were identified which were highly homogenous but had different amino acid sequence.

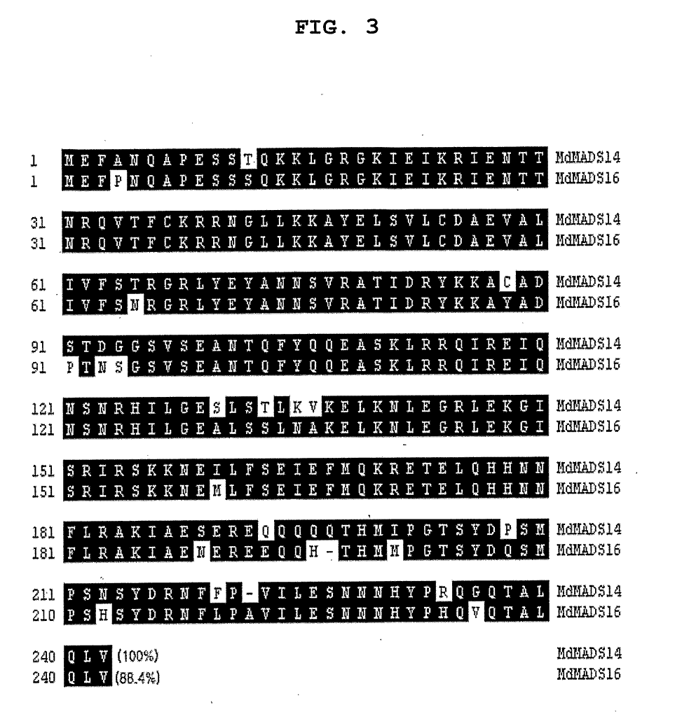

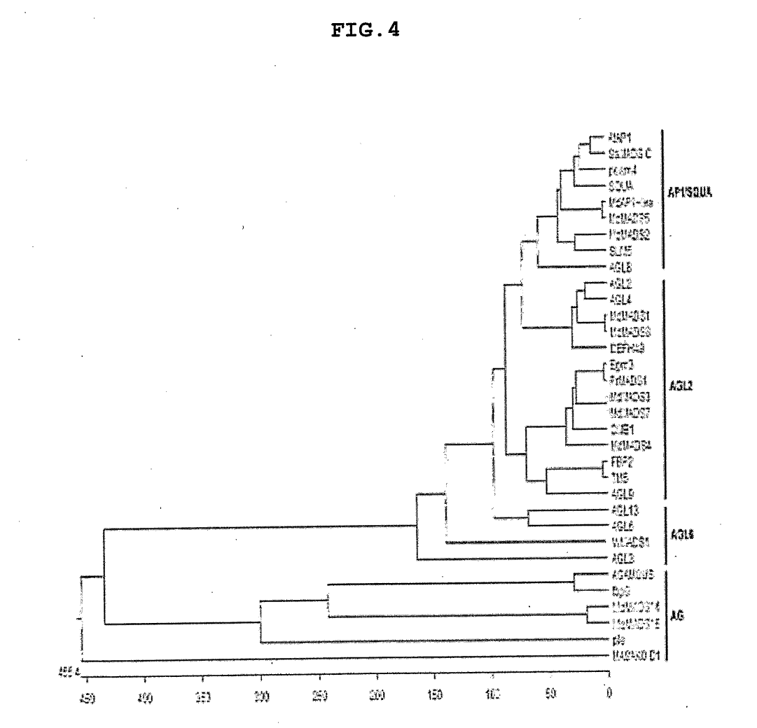

The full-length sequences thus obtained were each represented by SEQ. ID. No 1 (FIG. 1) and by SEQ. ID. No 2 (FIG. 2). Further, the nucleotide sequence set forth in SEQ. ID. No 2 were then analyzed by clustal method using MegAlign (DNAstar Inc., USA), a program for sequence analysis and was confirmed to be a novel gene having different nucleotide sequence from that represented by SEQ. ID. No 1. As shown in FIG. 1, the gene set forth in SEQ. ID. No.1 contains an open reading frame (ORF) encoding 242 amino acids, and also a nucleotide sequence encoding MADS-domain at its N-terminal region. Accordingly, it was designated ‘MdMADS14’. As shown in FIG. 2, the gene set forth in SEQ. ID. No 2, which was designated ‘MdMADS16’, also contains an ORF encoding 242 amino acids of the same size as the one in MdMADS14 gene, and also a nucleotide sequence encoding MADS-domain at its N-terminal region. The homology between MdMADS14 and MdMADS16 genes in 242 amino acid sequences was 88.4%, the homology between the two genes in MADS-domain region was 98.3%, and the homology between the two genes in the region other than MADS-domain was over 85% (FIG. 3). High homology of MADS-domain regions seems to be common among the members of MADS-box gene family, thus a gene having homology with MdMADS14 and MdMADS16 gene has to be identified based on the homology data from the region other than MADS-domain. Thus, it appears that MdMADA14 gene and MdMADS16 gene are members of the same family. Further it was found that, as shown by the phylogenetic tree of MADS-box genes (FIG. 4), these two genes are more closely related to each other than to any other gene of MADS-box gene group.

### EXAMPLE 2

### Analysis of Expression of MdMADS14 and MdMADS16 Genes in Various Organs and in Each Different Developmental Stage Using Real-Time PCR

Real-time PCR was performed to investigate the expressions of MdMADS14 gene and MdMADS16 gene in a variety of organs and in various developmental stages of fruits. Test samples were taken from various parts of an apple tree, i.e., leaves, flower buds, flowers, young fruit at 2 weeks after blooming and at 24 weeks after blooming. Total RNA was extracted from each of the above samples and used for real-time PCR was performed with it.

The expression levels of the genes in each organ and in developmental stage were quantified by real-time PCR using SYBR Green Master Mix Kit (Applied Biosystems, USA) as recommended by the supplier. Specifically, the first strand cDNA pool was constructed from 2 μg of total RNA extracted from each of the samples above. Primer concentrations were first optimized such that non-specific PCR products or a primer dimmer was not formed during the PCR. The optimum concentration determined for each primer was as follows: for MdMADS14 gene amplification, forward primer of SEQ. ID. No 10 and reverse primer of SEQ. ID. No 11 were 50 nM and 300 nM, respectively; for MdMADS16 amplification, forward primer of SEQ. ID. No 12 and reverse primer of SEQ. ID. No 13 were both 900 nM; and for actin gene amplification as an internal control, forward primer of SEQ. ID. No 14 and reverse primer of SEQ. ID. No 15 were both 900 nM.

Each primer set was designed such that the size of amplified products is not more than 120 bps for accurate measurement within a short reaction time possible. Real-time PCR was performed on the first strand cDNA pool from each organ and developmental stage using a primer set specific for each gene to be detected at optimum concentrations as described above. The PCR was carried out on a thermal cycler for real-time PCR (7300 Real-time PCR system, Applied Biosystems, USA) under the following cycling condition: initial incubation at 50° C. for 2 minutes to activate polymerase therein; followed by denaturation at 95° C. for 10 minutes; 40 cycles of denaturation at 95° C. for 15 seconds and hybridization or annealing at 60° C. for 1 minute. The signal intensity from fluorescent dye in each amplified product was detected in a hybridization or annealing step of every cycle of RT-PCR to quantify the PCR product amplified. The expressions levels of MdMADS14 gene and MdMADS16 gene in each organ and developmental stage were then determined and normalized against the amount of actin synthesized.

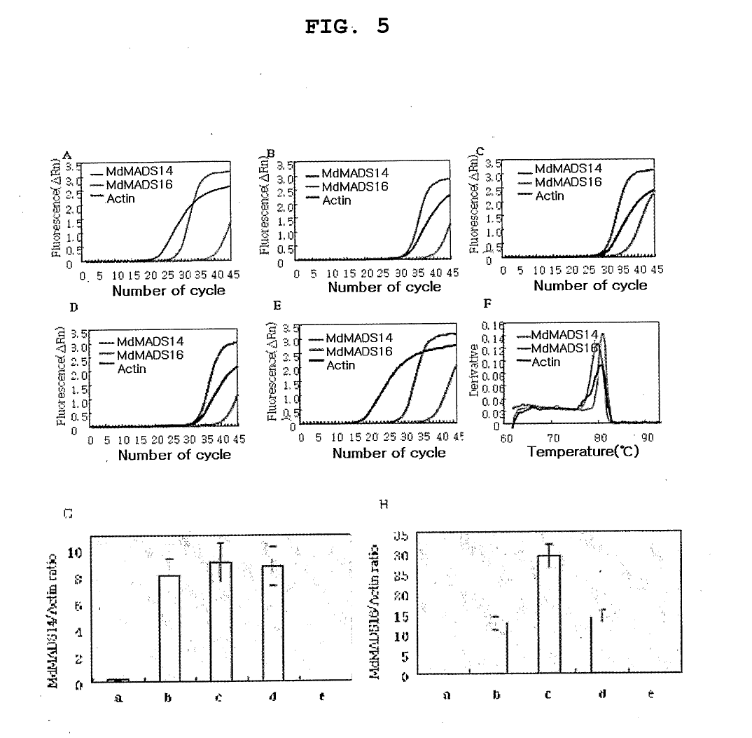

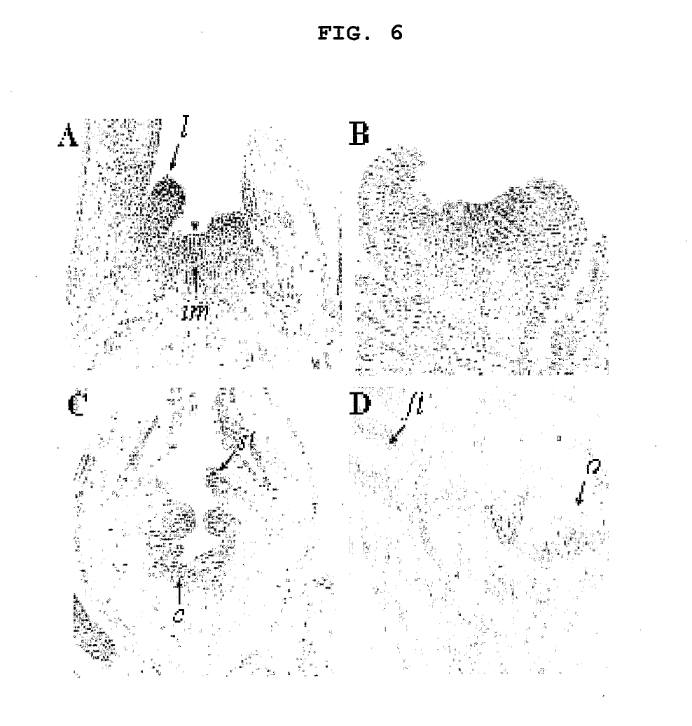

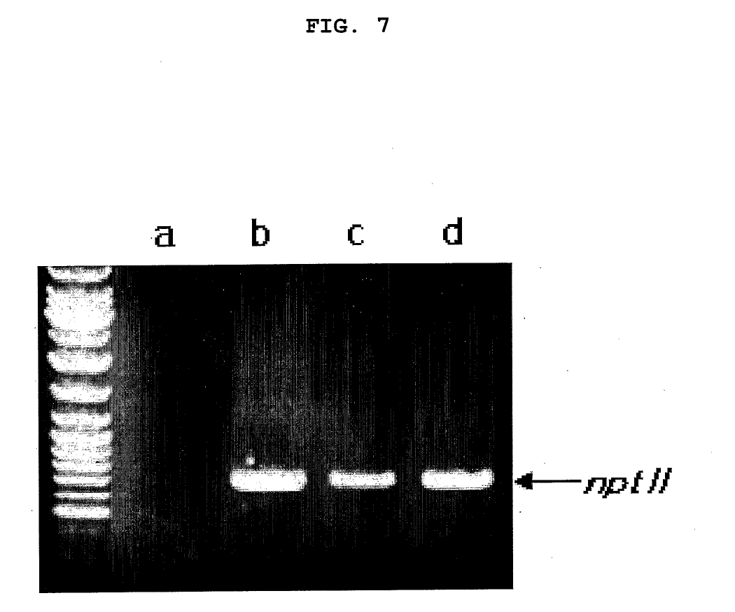

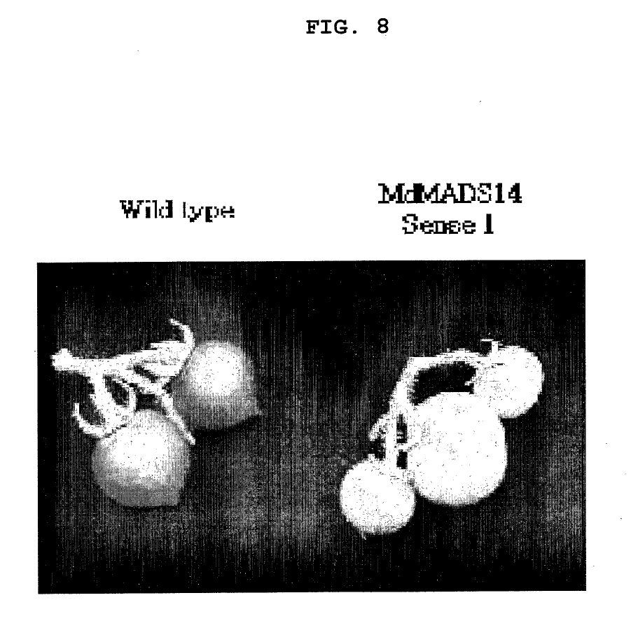

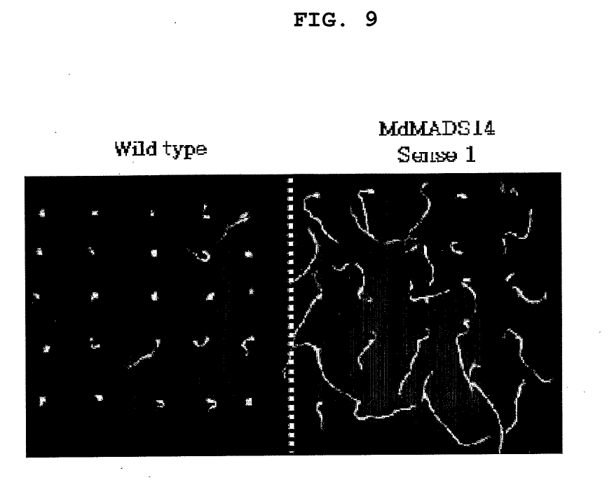

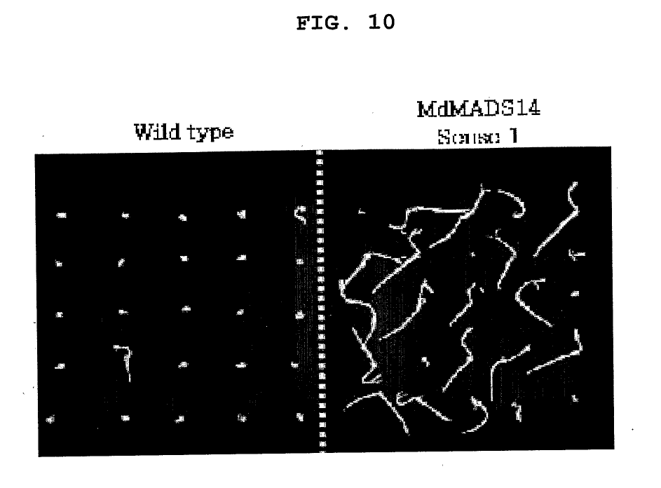

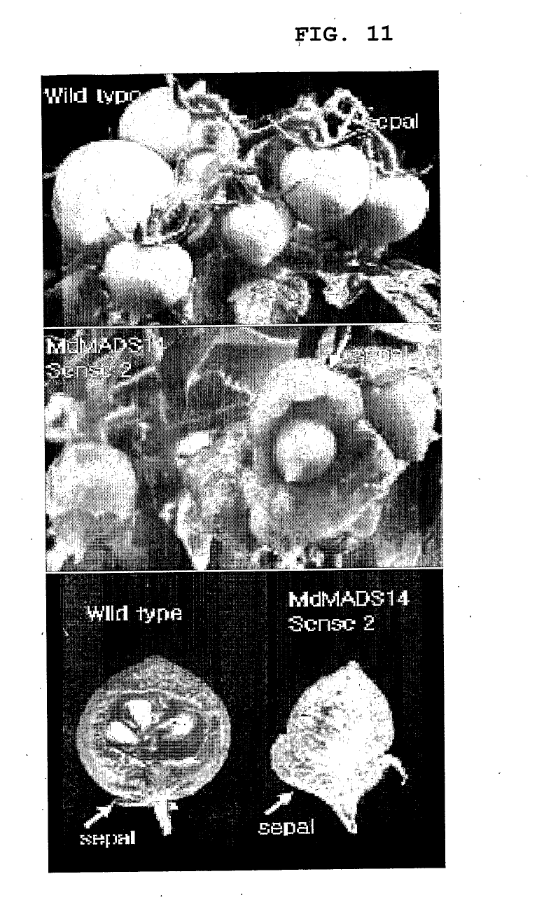

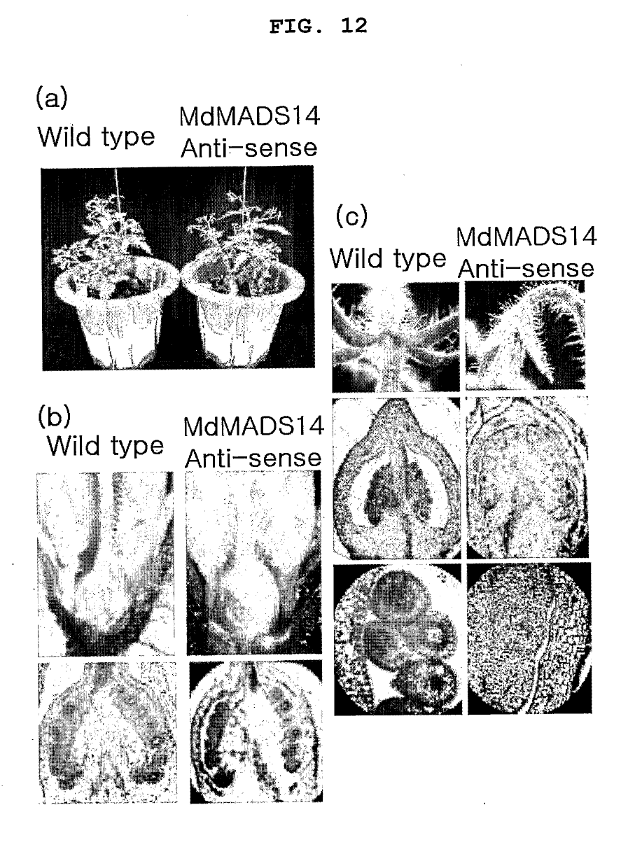

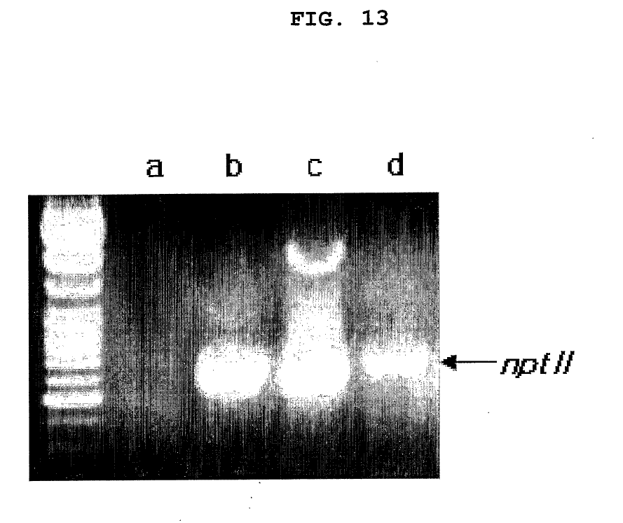

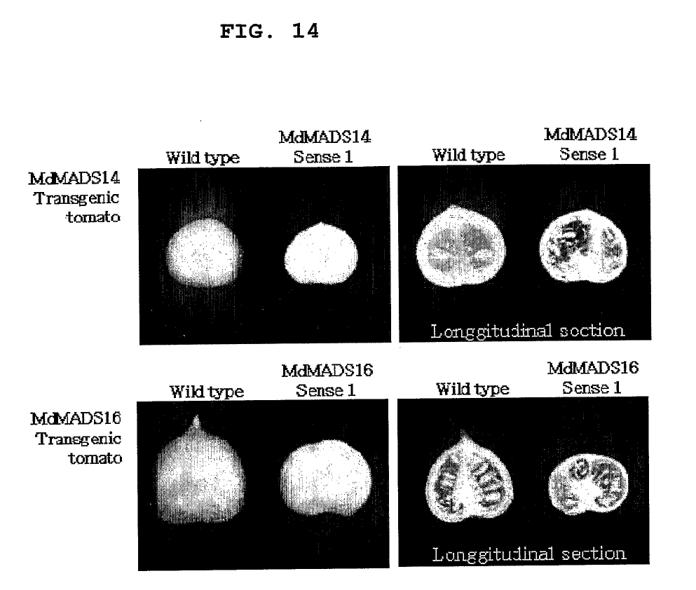

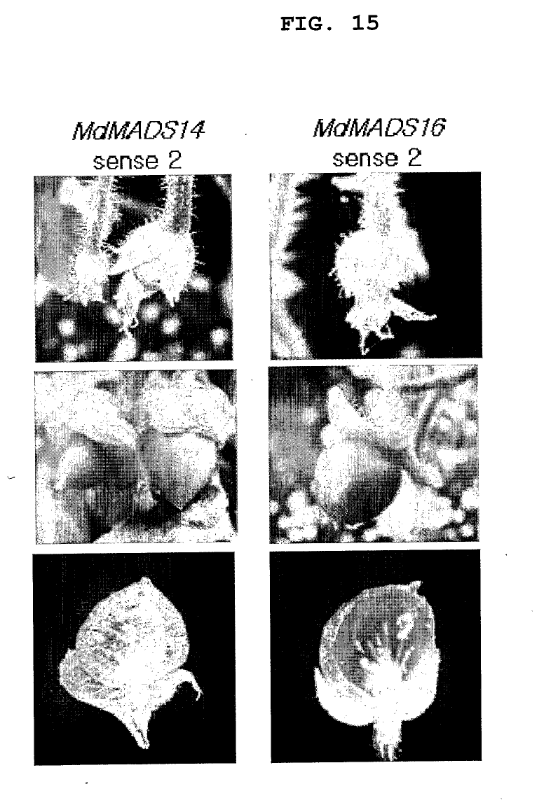

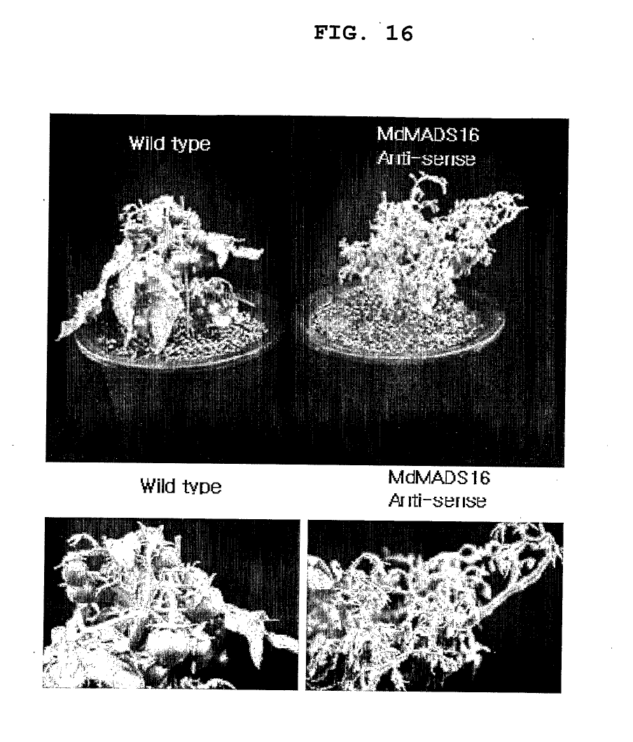

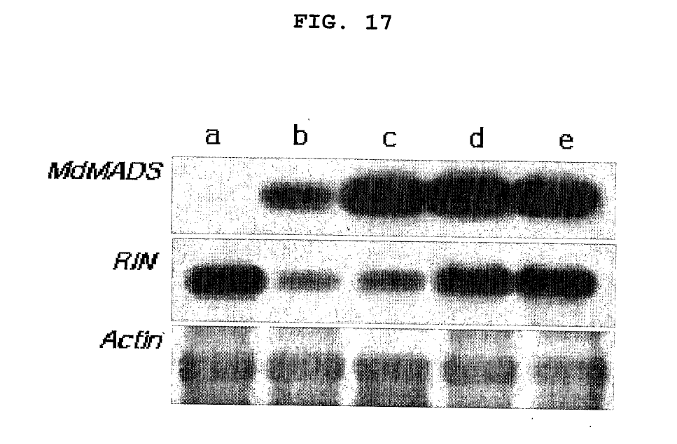

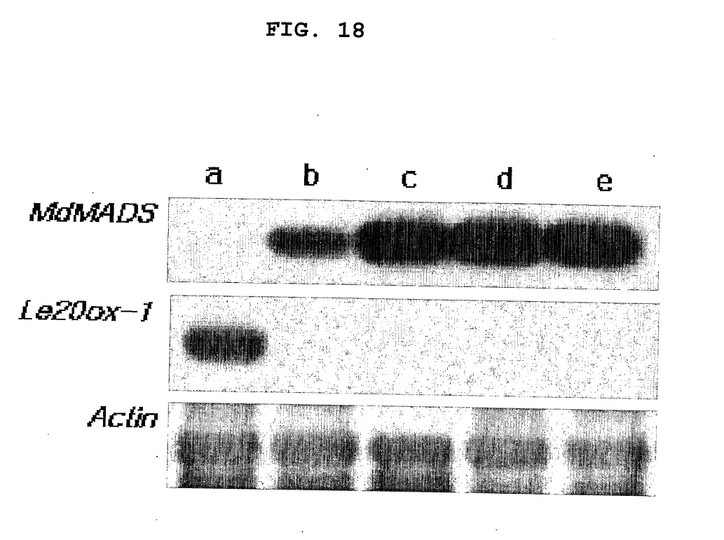

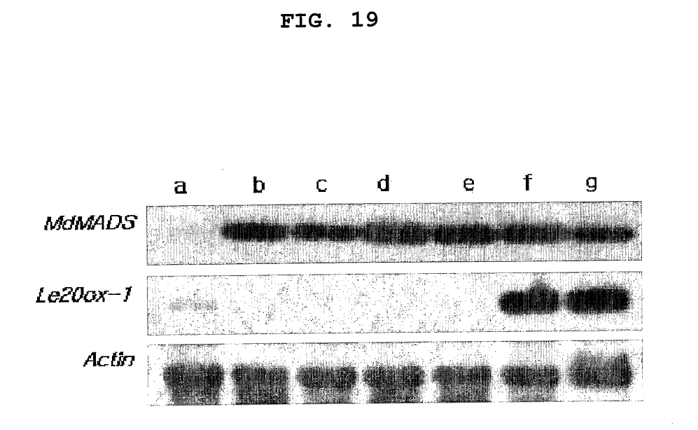

As shown in FIG. 5, the expression of MdMADS14 gene was observed from flower buds to the young fruit development stage, while it is rarely expressed in leaves and mature fruits of an apple tree. The expression pattern of MdMADS16 gene was similar to the one shown by MdMADS14 gene, only the expression levels of MdMADS16 gene were lower that of MdMADS14 gene. In order to examine specificity of each reaction product, melting curve analysis was performed. As a result, MdMADS14 and MdMADS16 were confirmed to be different from each other, showing different melting temperature.

### EXAMPLE 3

### mRNA Expression of MdMADS16 Gene by In Situ Hybridization

The expression of mRNA of MdMADS16 gene in young flower bud and flowers after pollination from an apple tree was analyzed by in situ hybridization. The sample cut at appropriate size was fixed in a fixing solution, and embedded in paraffin block. The paraffin block was then sectioned at 10 μm thick followed by hybridization with a appropriate nucleotide probe. The probe used herein was directed to a region other than MADS-domain and K-domain that are commonly found in MADS-box genes, and was prepared by labeling the antisense strand of MdMADS16 gene with DIG. The probe represented by SEQ. ID. No 16 was preferably used in this invention. However, the difference between the two genes was not distinguished by the probe, because homology in those regions between MdMADS16 gene and MdMADS14 gene was more than 88% (88.1%). In conclusion, as shown in FIG. 6, MdMADS16 gene was strongly expressed in inflorescence meristem and in ovule. The result indicates that MdMADS16 gene is involved in fruit and seed development and flower organ formation.

### EXAMPLE 4

### Generation of a Transgenic Plant Containing MdMADS14 Gene

DNA isolated from apple containing MdMADS14 gene as in Example 1 was digested with PstI and HindIII, then the resulting fragment was cloned into a pBluescriptSK(−) vector (Stratagene, USA) digested with the same enzymes.

A recombinant vector for over-expression was constructed by digesting it with Xba I and Cla I and then cloning forwardly into pGA1530 vector, a vector for plant transformation, pre-digested with the same enzymes. A recombinant vector for the inhibition of gene expression was also constructed by digesting recombinant pBluescriptSK(−) vector containing MdMADS14 gene with Xba I and HindIII, and cloning it reversely into pGA1530 vector, a vector for plant transformation, pre-digested with the same enzymes. The pGA1530 vector for plant transformation contains 35S promoter, T7 terminator and nptII (neomycin phosphotransferase) as a selection marker which provides kanamycin resistance (Plant molecular Biology Manual, 1988, A3:1-19). The resulting recombinant plasmid containing MdMADS14 gene in forward and reverse direction was designated ‘pMdMADS14’, and ‘pMdMADS14-R’, respectively. The recombinant plasmids were then used to transform Agrobacterium tumefaciens LBA4404 (A. Hoekema et al., 1983, Nature, 303, 179-181), and each transformed Agrobacterium tumefaciens containing ‘pMdMADS14’ or ‘pMdMADS14-R’ was selected in a growth medium containing Kanamycin, which were deposited with Korean Collection for Type Cultures (KCTC) of Korea Institute of Bioscience and Biotechnology (KRIBB) on Jan. 30, 2004 (Accession No: KCTC 10588BP).

Minitomato (Lycopersicon esculentum cv. ‘Micro-Tom’) was used for the generation of a transgenic plant into which pMdMADS14 was transferred. In order to select a target tissue, mature seeds of minitomato were first sterilized by immersion in 70% ethanol for 1 minute, washing with sterilized water three times, immersion in 2% NaOCl (Sodium hypochloride) for 15 minutes, followed by washing with sterilized water more than 7 times. The seeds were then transferred to a plate containing seed germination medium (½ MS, 3% sucrose, 0.8% agar). 14 days later, seeds began to be germinated. As cotyledon was opened and true leaf began to be sprout, the cotyledon was selected as a target tissue for transformation.

The recombinant Agrobacterium tumefaciens LBA4404, prepared as in the above Example, was cultured in YEP medium (1% yeast extract, 1% peptone, 0.5% NaCl) containing 50 mg/e of kanamycin until OD₆₀₀ was 0.8. The cultured Agrobacterium was co-cultured in ½ MS medium supplemented with 200 μM of acetosyringone with shaking at 150 rpm, at 22° C. for 2 hours. Then, the target tissue, cotyledon section of a minitomato prepared as above, was added thereto and co-cultured with shaking at 150 rpm, at 22° C. for 10 minutes. The resulting cotyledon sections were then placed onto a plate containing co-culture medium (MS, IAA 1 μM, zeatin 10 μM, sucrose 3%, acetosyringone 200 μM, agar 0.7%) and cultured for 2 days. Then, the cotyledon sections were transferred onto a regeneration medium (MS, IAA 1 μM, zeatin 10 μM, sucrose 3%, cefotaxime 350 mg/l, kanamycin 50 mg/l, agar 0.7%), followed by two sub-cultures in succession at three weeks interval. Then, shoots generated from the cotyledon sections were transferred onto selective root-inducing medium (MS ½, IAA 1 μM, sucrose 3%, kanamycin 50 Mg/l, agar 0.7%) containing kanamycin to select transformants. The transformed shoots can be differentiated from untransformant since the untransformant undergoes necrosis accompanied by color change while transformed shoots were growing normally with roots. Shoots with roots were then transferred onto soil after acclimatization.

As a result, transformants over-expressing MdMADS14 gene (senses) showed two phenotypes, each of which was designated “transformant 1” (MdMADS14 sense 1) and transformant 2 (MdMADS14 sense 2). Another transformant in which the expression of MdMADS14 gene was suppressed (anti-sense) showed the phenotype of no-fruit formation, which was designated “inhibited transformant 1” (MdMADS14 anti-sense). As shown in FIG. 7, the successful integration of MdMADS14 gene into the genome of each of the above transformant was verified by PCR amplifying nptII gene in each transformant. A set of forward and reverse Primers used for the PCR to confirm the integration of nptII gene is represented by SEQ. ID. No 17 and by SEQ. ID. No 18, respectively.

### EXAMPLE 5

### Analysis of the Functions of MdMADS14 Gene in a Transgenic Plant

Phenotypes of MdMADS14 senses and anti-sense, prepared in the above example 4, were investigated to analyze the functions of MdMADS14 gene in plants.

As a result, MdMADS14 senses showed two different phenotypes; sense 1 showed delayed ripening but promoted seed germination and sense 2 showed the characteristics of that sepal was changed into fruit flesh and parthenocarpic fruit was formed.

As shown in FIG. 8 and FIG. 9, sense 1 showed delayed fruit ripening, comparing to wild type, but promoted seed germination. Such characteristics were observed not only in T1 generation but also in T2 generation, indicating that the characteristics are inherited.

In order to confirm the above result, germination speed and germination rate of seed (T2 generation) of sense 1 were measured (Table 1, measurement of the primary germination rate). Specifically, 25 seeds each from wild type tomato and MdMADS14 sense 1 as shown in FIG. 8 were harvested, and then dried for 10 days. Subsequently, the seeds were placed on a filter paper moisten with distilled water in a petridish and cultured at 25° C. for 5 days. Germination speed for each group was measured and statistically analyzed. As shown in FIG. 9, seed germination in sense 1 was much promoted than in wild type. As shown in Table 1, while the average germination speed of seeds in sense 1 was 1.77 day, the average germination speed of seeds in wild type was 3.8 days. Moreover, germination rate of seed in sense 1 was twice as high as the wild type. Wild type seeds and seeds (T2 generation) of sense 1 were stored in cool/dry condition for a year, then germination speed and germination rate were investigated again by the same method as used above (Table 2, measurement of the second germination rate). As a result, as shown in FIG. 10, seed germination speed and germination rate in sense 1 was still promoted, comparing to wild type. As shown in Table 2, the average germination speed of sense 1 seeds was 3 days, while the average germination speed of wild type tomato seeds was 6 days, indicating that germination speed was shortened in sense 1 twice as much as in wild type, and germination rate in sense 1 was still two-fold higher than in wild type. After one-year storage, germination speed and germination rate were slow and dull in general, but the speed and rate in sense 1 were still about two-fold higher than those in wild type.

TABLE 1

| | Seeds from | Seeds from |
|---|---|---|
| | Transformant 1 (T2) | non-transformant |
| Average Germination Term | 1.77 Days | 3.8 Days |
| Germination Rate | 100% | 50% |

TABLE 2

| | Seeds from | Seeds from |
|---|---|---|
| | Transformant 1 (T2); | Non-transformant; |
| | 1 year storage in | 1 year storage in |
| | a cool/dry place. | a cool/dry area |
| Average Germination Term | 3 Days | 6 Days |
| Germination Rate | 92% | 40% |

MdMADS14 sense 2 showed such phenotype as sepal was changed into fruit flesh and parthenocarpic fruit was formed. As shown in FIG. 11, sepal in sense 2 was changed into fruit flesh with being corpulent and parthenocarpic fruit was formed without seed formation, unlike wild type.

In the meantime, MdMADS14 anti-sense plant showed common phenotype that was characterized by no-fruit formation. As shown in FIG. 12, the development of floral organ in the early stage was not affected (FIG. 12(a) and FIG. 12(b)) but seed development after pollination was inhibited, and so fruits were not grown enough (FIG. 12(c)). Floral organ and ovary of each developmental stage in anti-sense were fixed in a fixing solution (50 mM PIPES; ph 6.8, 4% paraformaldehyde), leading to plastic embedding. The samples were cut by 7 μm and stained with toluidine blue O, followed by histological observation.

As a result, as shown in FIGS. 12(b) and 12(c), the development of ovule in early developmental stage of floral organ was not much affected, but the development of ovule and ovary after pollination was clearly inhibited.

### EXAMPLE 6

### Generation of a Transgenic Plant Having MdMADS16 Gene and Analysis of the Functions of MdMADS16 Gene in the Plant

DNA isolated from apple containing MdMADS16 gene as in Example 1 was digested with PstI and HindIII and the resulting fragment was cloned into a pBluesecriptSK(−) vector (Stratagene, USA) digested with the same enzymes. The vector was digested with BamHI and HindIII again, followed by fill-in with the fragment containing the above gene. Then, MdMADS16 gene was sub-cloned into pRTL2 vector (Restrepo, M. et al., Plant Cell, 1990, 2: 987-998) pre-digested with Sma I. The plasmid prepared by the above procedure included both forward cloning and reverse cloning of MdMADS16 gene, so both forwardly cloned MdMADS16 gene and reversely cloned MdMADS16 gene were separated through nucleotide sequencing. Each of them was digested with HindIII, followed by cloning into pCAMBIA 2301 vector (CAMBIA, Australia) for plant transformation, which was pre-digested with the same enzyme. pCAMBIA 2301 vector for plant transformation contains 35S promoter, nos (nopaline synthase) and nptII (neomycin phosphotransferase) as a marker gene, which provides kanamycin resistance. The recombinant plasmid in which MdMADS16 was forwardly inserted was designated ‘pMdMADS16’ and the recombinant plasmid in which MdMADS16 gene was reversely inserted was designated ‘pMdMADS16-R’. Those recombinant plasmids were inserted in Agrobacterium tumefaciens LBA4404, and then strains containing those recombinant plasmids, pMdMADS16 and pMdMADS16-R, were selected from kanamycin selection medium. The selected Agrobacterium tumefaciens containing the recombinant plasmid pMdMADS16 was deposited at Korean Collection for Type Cultures (KCTC) of Korea Institute of Bioscience and Biotechnology (KRIBB) on Jan. 30, 2004 (Accession No: KCTC 10589BP), which was further used for Agrobacterium tumefaciens-mediated transformation of minitomato plant as described in Example 3. MdMADS16 senses, transgenic minitomato plant generated by the above procedure, showed two phenotypes and was designated MdMADS16 sense 1 and MdMADS16 sense 2 depending on their phenotypes. The phenotypes were the same as those of MdMADS14 senses. In other words, MdMADS16 sense 1, as shown in FIG. 14, showed delayed ripening but promoted seed germination like MdMADS14 sense 1. MdMADS16 sense 2, as shown in FIG. 15, showed the phenotype of transformation of sepal into fruit flesh as shown in FIG. 16 like MdMADS14 sense 2. MdMADS16 anti-sense showed phenotype of no-fruit formation after pollination, like MdMADS14 anti-sense, which indicate that both MdMADS14 gene and MdMADS16 genes are involved in fruit formation.

From the Examples above, it can be known that MdMADS14 gene and MdMADS16 gene belong to the same family, showing high homology, and are involved in seed and fruit development from the functional analysis of transgenic plants. Such functions of MdMADS14 and MdMADS16 gene of the present invention are novel, which has not been found previously in currently known MADS-box genes.

### EXAMPLE 7

### Analysis of the Expressions of RIN Gene in MdMADS14 and MdMADS16 Senses by RT-PCR

Both MdMADS14 sense 1 and MdMADS16 sense 1 generated in Example 4 and Example 6, respectively, show the same phenotype as was shown for the tomato plant in which the ethylene synthesis is inhibited during ripening (Julia Vrebalov et al., SCIENCE, 2002, 296: 343-346). Thus, RIN gene expression that plays an important role in ethylene synthesis during ripening of tomato was investigated in MdMADS14 sense 1 and MdMADS16 sense 1 by reverse transcription-polymerase chain reaction (RT-PCR).

Specifically, the first strand cDNA pool was prepared from each 2 μg of total RNA extracted from fruits of senses and wild type using reverse transcriptase primed with oligo(dT) primer. RT-PCR was then performed by using the cDNA pool as a template according to the method known in the pertinent art. Particularly, the PCR conditions were set to assure the validity of the analysis such that the amplified products are in exponential phase. Actin primer, used in the above example 2, was used for internal control.

In order to confirm whether or not an introduced gene was expressed in a transformant, forward primer represented by SEQ. ID. No 19 and reverse primer represented by SEQ. ID. No 20 were prepared based on common nucleotide sequence region (referred as MdMADS) between MdMADS14 gene and MdMADS16 gene, and then used for the amplification of MdMADS. Forward primer represented by SEQ. ID. No 21 and reverse primer represented by SEQ. ID. No 22 were used for the amplification of RIN gene. PCR was performed under the following cycling condition: initial denaturation at 95° C. for 10 minutes; 25 cycles of denaturation at 95° C. for 30 seconds, annealing at 55° C. for 1 minute and extension at 72° C. for 1 minute; and final extension at 72° C. for 10 minutes. The obtained PCR products were then electrophoresed on an agarose gel, and the gel was transferred onto a Hybond N+ membrane according to a method known in the pertinent art. The membrane was then allowed to hybridize with the probe for RIN gene, MdMADS gene and actin gene, each of which was labeled with biotin, and the signals from each probe hybridized to its corresponding target sequence were detected using Southern-Star™ System (Applied biosystems, USA) recommended by the supplier.

The results are shown in FIG. 17. First, the successful expression of the gene introduced into each transformation was confirmed by the signals detected only in transformants but not in a non-transformant. Further, the expression of RIN gene was reduced in MdMADS14 sense 1 and MdMADS16 sense 1, which is consistent with delayed ripening. On the contrary, the expressions of RIN gene in MdMADS14 sense 2 and MdMADS16 sense 2 that forms parthenocarpic fruit were not much different from that of wild type.

It has been known that reduced expression of RIN gene represents the reduced ethylene synthesis during ripening since RIN gene plays an essential role in ethylene synthesis during ripening (R. C. Herner et al., Plant Physiol., 1973, 52: 38-42; Julia Vrebalov et al., SCIENCE, 2002, 296: 343-346). Therefore, the results from this experiment indicate that the delayed ripening in both MdMADS14 sense 1 and MdMADS16 sense 1 was resulted from the decrease of ethylene synthesis by the introduction of MdMADS14 and MdMADS16 gene. Further, it indicates that MdMADS14 gene and MdMADS16 gene of the present invention can be effectively used for the regulation of fruit development and fruit ripening delay.

### EXAMPLE 8

### Analysis of the Expression of Le20ox-1 Gene in a Transformant Harboring MdMADS14 gene or MdMADS16 Gene by RT-PCR

In order to examine the level of active gibberellin synthesized in MdMADS14 senses and MdMADS16 senses obtained in Example 4 and Example 6, the expression level of Le20ox-1 gene was investigated. As mentioned hereinbefore, the mRNA synthesis of Le20ox-1 gene, which is involved in the conversion of C-20 into C-19 gibberellin followed by activation by 3β-hydroxylase leading to active gibberellin, is regulated by negative feedback from high level of active gibberellin present in a cell. Thus, the mRNA level of Le20ox-1 gene-is negatively correlated with the amount of active gibberellin present.

The expression levels of Le20ox-1 gene in fruits and leaves from transgenic plants and wild type were compared and determined by RT-PCR followed by Southern blot as described in Example 7 except that, for the amplification of Le20ox-1 gene, forward primer represented by SEQ. ID. No 23 and reverse primer represented by SEQ. ID. No 24 were used.

The results are shown in FIG. 18. The expression of Le20ox-1 gene in fruits of MdMADS14 senses and MdMADS16 senses were significantly decreased, compared with that of wild type, which suggests that synthesis of active gibberellin in MdMADS14 senses and MdMADS16 senses was much increased, comparing to that in wild type.

The expression levels of Le20ox-1 gene in leaves of MdMADS14 senses, MdMADS16 senses, MdMADS14 anti-sense and MdMADS16 anti-sense were also compared. The results are shown in FIG. 19. The expression of Le20ox-1 gene were significantly decreased in leaves of MdMADS14 senses and MdMADS16 senses, compared with wild type, but the expression of Le20ox-1 gene were increased in leaves from MdMADS14 anti-sense and MdMADS16 anti-sense. The results indicate that synthesis of active gibberellin in MdMADS14 senses and MdMADS16 senses was much increased, but synthesis of active gibberellin in MdMADS14 anti-sense and MdMADS16 anti-sense was much decreased, comparing to that in wild type.

Therefore, MdMADS14 gene and MdMADS16 gene of the present invention can be effectively used for the regulation of active gibberellin synthesis.

### INDUSTRIAL APPLICABILITY

The genes of the present invention regulate fruit and seed development by inhibiting or promoting active gibberellin synthesis. A transgenic plant containing the gene of the present invention exhibits phenotype that is characterized by promoted seed development, transformation of calyx into fruit flesh and formation of parthenocarpic fruit. Thus, the genes of the present invention can be effectively used for the regulation of active gibberellin synthesis, production of parthenocarpic fruit, and fruit and seed development, all of which are important in increasing the productivity of crops.

[Sequence List Text]

The SEQ. ID. No 1 is a nucleotide sequence of MdMADS14,

The SEQ. ID. No 2 is a nucleotide sequence of MdMADS16,

The SEQ. ID. No 3, No 4 and No 5 are nucleotide sequences of degenerate primers used for degenerate PCR described in Example 1,

The SEQ. ID. No 6 and No 7 are nucleotide sequences of primers used for PCR to amplify the nucleotide sequence of SEQ. ID. No 1 in Example 1,

The SEQ. ID. No 8 and No 9 are nucleotide sequences of primers used for PCR to amplify the nucleotide sequence of SEQ. ID. No 1 in Example 2,

The SEQ. ID. No 10 and No 11 are nucleotide sequences of primers used for the amplification of MdMADS14 gene by real-time PCR and RT-PCR described in Example 2,

The SEQ. ID. No 12 and No 13 are nucleotide sequences of primers used for the amplification of MdMADS16 gene by real-time PCR and RT-PCR described in Example 2,

The SEQ. ID. No 14 and No 15 are nucleotide sequences of primers used for the amplification of actin gene by real-time PCR and RT-PCR described in Example 2,

The SEQ. ID. No 16 is a nucleotide sequence of probe used in in situ hybridization described in Example 3,

The SEQ. ID. No 17 and No 18 are nucleotide sequences of primers used for PCR to confirm the expression of nptII gene described in Example 4,

The SEQ. ID. No 19 and No 20 are nucleotide sequences of primers used for real-time PCR and RT-PCR for the amplification of MdMADS gene described in Example 7,

The SEQ. ID. No 21 and No 22 are nucleotide sequences of primers used for real-time PCR and RT-PCR for the amplification of RIN gene described in Example 7,

The SEQ. ID. No 23 and No 24 are nucleotide sequences of primers used for real-time PCR and RT-PCR for the amplification of Le20ox-1 gene described in Example 8.

Those skilled in the art will appreciate that the conceptions and specific embodiments disclosed in the foregoing description may be readily utilized as a basis for modifying or designing other embodiments for carrying out the same purposes as the present invention. Those skilled in the art will also appreciate that such equivalent embodiments do not depart from the spirit and scope of the invention as set forth in the appended claims.

1. An isolated gene regulating fruit and seed development selected from a group consisting of a gene having a nucleotide sequence set forth in SEQ. ID. No 1 containing a nucleotide sequence encoding MADS-domain, a gene having a nucleotide sequence set forth in SEQ. ID. No 2 containing a nucleotide sequence encoding MADS-domain and a gene encoding an amino acid sequence having at least 85% homology within the region other than MADS-domain.

2. An expression vector comprising the gene according to claim 1.

3. The expression vector according to claim 2 wherein the expression vector is pMdMADS14 into which a gene having the nucleotide sequence set forth in SEQ. ID. No 1 is inserted in forward direction (Accession No: KCTC 10588BP).

4. The expression vector according to claim 2 wherein the expression vector is pMdMADS16 into which a gene having the nucleotide sequence set forth in SEQ. ID. No 2 is inserted in forward direction (Accession No: KCTC 10589BP).

5. A transgenic plant cell containing the gene according to claim 1.

6. A transgenic plant whose fruit and seed development is regulated, and that is prepared by regeneration of the transgenic plant cells according to claim 5 by tissue culture technique.

7. The transgenic plant according to claim 6 wherein the plant is selected from a group consisting of food crops such as rice, wheat, barley, corns, soybean, potato, red bean, oat, sorghum; vegetables such as Chinese cabbage, radish, red pepper, strawberry, tomato, watermelon, cucumber, cabbage, melon, pumpkin, spring onion, onion, carrot; industrial crops such as ginseng, Acanthopanax senticosus, tobacco, cotton, sesame, sugar cane, sugar beet, Perilla japonica, peanut, rape; fruits such as apple, pear, orange, jujube, peach, kiwifruit, grapes, tangerine, persimmon, plum, apricot, bananas; floricultural crops such as rose, gladiolus, gerbera, carnation, chrysanthemum, lily, tulip; forage crops such as ryegrass, red clover, orchard grass, alfalfa, tall fescue, perennial ryegrass; fiber crops such as cotton plant; and landscape plants such as flowers and shrubs.

8. An offspring or a clone of a transgenic plant according to claim 6.

9. A Fruit, seed, ear, tuber, tuberous root, column, callus or a protoplast of a transgenic plant according to claim 6.

10. The transgenic plant according to claim 6 wherein the plant shows one of the following phenotypes:
a phenotype in which sepal is transformed into fruit flesh and parthenocarpic fruit is formed; a phenotype in which seed development is promoted and ripening is delayed; and a phenotype in which fruit and seed development is inhibited.

11. A method of preparing a transgenic plant whose fruit and seed development was regulated, comprising the steps of:
1) Constructing an expression vector comprising the gene according to claim 1;
2) Transferring the vector constructed in Step 1) into Agrobacterium;
3) Co-culturing the transformed Agrobacterium of step 2) with plant tissue; and
4) Regenerating the transformed tissue into a mature transgenic plant.

12. A Composition for fruit and seed development in a plant comprising the gene according to claim 1 or the expression vector according to any one of claims 2-4 as an effective ingredient.

13. A Composition for regulating the synthesis of active gibberellin containing the gene according to claim 1 or the expression vector according to any one of claims 2-4 as an effective ingredient.
